(19) United States
(12) Patent Application Publication
(10) Pub. No.: US 2003/0215409 A1
(43) Pub. Date: Nov. 20, 2003

(54) Process for the treatment of human keratinous fibers

(75) Inventors: Francis Xavier Quinn, Paris (FR); Francis Pruche, Senlis (FR); Yann Mahe, Morsang Sur Orge (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/395,337

(22) Filed: Mar. 25, 2003

Related U.S. Application Data
(60) Provisional application No. 60/374,806, filed on Apr. 24, 2002.
(60) Provisional application No. 60/429,539, filed on Nov. 29, 2002.

(30) Foreign Application Priority Data
Mar. 25, 2002 (FR) 0203710
Nov. 28, 2002 (FR) 0214973

(51) Int. Cl. A61K 7/06

(52) U.S. Cl. 424/70.1

(57) ABSTRACT

The invention relates to a process for densification of the hair, comprising a stage of application to the hair and/or the scalp of a composition consisting, in a physiologically acceptable medium, of an effective quantity of at least one precursor of densification chosen from among the compounds comprising at least one aromatic cycle having at least two hydroxyl groups borne by two consecutive carbon atoms of the aromatic cycle and an effective quantity of a catalytic system comprising one first constituent chosen from among the salts and oxides of Mn(II) and/or Zn(II) and mixtures thereof and a second constituent chosen from among the alkaline hydrogenocarbonates, the alkaline-earth hydrogenocarbonates and mixtures thereof.

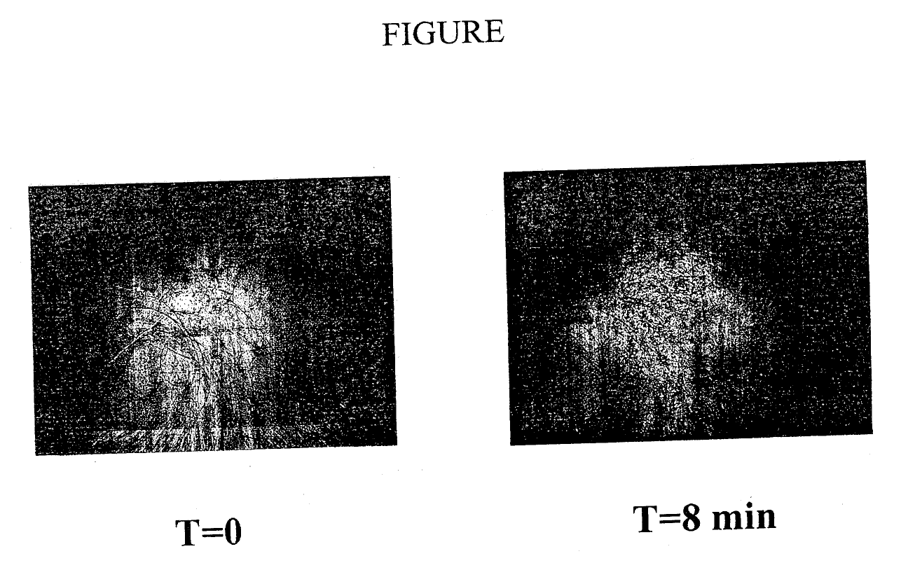

It also relates to a composition containing, in a physiologically acceptable medium, the said precursor of densification and catalytic system, combined with an active principle promoting the regrowth of hair, with delayed effect or for the dressing of the hair.

### REFERENCE TO PRIOR APPLICATIONS

This application claims priority to US provisional application ______(corresponding to French application 0214973) filed Nov. 29, 2002, and to U.S. provisional application No. 60/374,806 filed Apr. 24, 2002, as well as to French patent applications 0203710 filed Mar. 25, 2002, and 0214973 filed Nov. 28, 2002, all of which are incorporated herein by reference.

### SUMMARY OF THE INVENTION

This invention relates to a process for the treatment of human keratinous fibers, preferably the immediate densification of balding areas, and to the use of the combination of a precursor of densification and a mild catalytic system as an agent for densification of keratinous fibers and in particular of the hair of human beings. It also relates to a (cosmetic or dermatological) composition containing this precursor-catalytic system combination in the presence of an active principle promoting the regrowth of the keratinous fibers and/or limiting loss thereof, with delayed effect or for the dressing of the hair.

### BACKGROUND OF THE INVENTION

The growth of the keratinous fibers and more specifically of the hair and the renewal thereof are determined principally by the activity of the hair follicles and of their matrix environment. Their activity is cyclical and is composed essentially of three phases, that is, the anagenic phase, the catagenic phase and the telogenic phase.

The anagenic phase (active or growth phase), which lasts for several years and during which the hair grows longer, is followed by a very short and transitory phase which lasts for a few weeks. In the course of this phase, the hair undergoes an evolution, the follicle atrophies and its dermal implantation appears increasingly high.

The terminal phase or telogenic phase which lasts for a few months, corresponds to a resting phase for the follicle and the hair eventually falls out. At the end of this resting period, a new follicle is regenerated in situ and another cycle begins again.

The hair, therefore, is renewed on an ongoing basis, and out of the approximately 150,000 hairs which make up a head of hair, about 10% are at rest and will be replaced in a few months.

The natural falling out or loss of hair may be estimated, on the average, at some one hundred hairs per day for a normal physiological state. This process of ongoing physical renewal undergoes a natural evolution in the course of aging, the hairs become finer and their cycles shorter.

In addition, different causes can lead to a significant temporary or permanent loss of hair. It may be a matter of loss and deterioration of the hair following pregnancy (post partum), during states of undernourishment or dietary imbalances or else during states of asthenia or hormonal dysfunction as may be the case during or following menopause. It also may be a matter of loss or deterioration of the hair in relation to seasonal phenomena.

It likewise may be a matter of alopecia, which is due essentially to a disturbance of hair renewal leading at the outset to acceleration of the frequency of cycles to the detriment of the quality of the hair, then the quantity thereof. The successive growth cycles end up in increasingly fine hair and increasingly short hair, little by little becoming a non-pigmented down. Areas are affected preferentially, in particular the temporal or frontal spaces in men and, in women, a diffuse alopecia of the vertex is noted.

The term alopecia also covers a whole family of hair follicle damage ultimately resulting in the permanent, partial or general loss of hair. Most especially, it is a matter of androgenic alopecia. In a substantial number of cases, early hair loss occurs in genetically predisposed subjects; it then is a matter of androchrono-genetic alopecia; this form of alopecia concerns men in particular.

Furthermore, it is known that certain factors such as a hormonal imbalance, a physiological stress, malnutrition, can accentuate the phenomenon.

In certain inflammatory-type scalp dermatoses such as, for example, psoriasis or seborrheic dermatitis, the loss of hair may be highly accentuated or lead to highly disturbed follicle cycles.

For many years, compositions making it possible to eliminate or to reduce alopecia, and in particular to induce or stimulate the growth of hair or to decrease loss thereof or for dressing the hair, have been sought in the cosmetic or pharmaceutical industry.

The compositions currently used have the drawback of not bringing about the densification of the hair for several days, or even several weeks, after the treatment, which for certain individuals may be very bothersome or even handicapping. Thus, for finasteride it is necessary to wait from 9 to 12 months to perceive a benefit, for minoxidil or aminexil, the user begins to have a benefit after 1.5 months and more generally at the end of 3 to 6 months. Now, to date there are no compositions for immediate hair densification.

Furthermore, the active principles with prolonged action generally are applied daily, or sometimes even twice daily, which gives rise to a very considerable wait on the part of the user who sometimes, seeing no effect after the use of his first bottle or his first prescription, does not repeat the application further. Then it is said that the “observance” (that is, the following of recommendations for use in terms of dose and duration) is not good.

Moreover, embarking on a hair restoration strategy requires that a threshold of tolerance for his balding condition be exceeded. He therefore needs an immediately visible and perceptible effect. This furthermore is most especially suited to the current needs of the users in terms of ease and speed of accomplishment of the treatment.

The hair products for setting of the hair most widespread on the cosmetic market are compositions for spraying in an aerosol can or in a pump bottle, such as lacquers, sprays or mousses, consisting essentially of a solution more often than not alcoholic or hydroalcoholic and of a filmogenic polymer soluble in water or in alcohol, in combination with various cosmetic additives.

Numerous compositions of the prior art for holding and/or styling of the hair, which contain hairdressing polymers in their formulation, can damage the cosmetic properties of the hair. The latter, after application of such compositions, can become rough, lose its natural softness or its pleasing appearance.

Therefore for hair compositions, in particular for the holding and/or setting of the hairstyle, polymers are sought which impart to the hair, in addition to a satisfactory holding and/or setting, good cosmetic properties, in particular good untangling, softness and a pleasing feel.

Nonetheless, the use of these polymers has several drawbacks. The first lies in the fact that, when the polymers are used above a certain concentration, the compositions are difficult to apply (the viscosity increases). That creates excesses in certain places on the hair and therefore cosmetic flaws, and that also implies that certain portions of the hair receive less composition which, in the end, results in a lesser effect on these portions.

The second drawback lies in the fact that they sometimes are difficult to implement. As a matter of fact, the polymeric compounds with low solubility in water require the use of an organic solvent or a mixture of organic solvents. That creates several collateral problems, in particular an environmental problem, an effect on the cosmetic quality of the hair. Certain polymeric compounds may be used in water without addition of any solvent at all. In these cases, the limitation lies in the fact that these polymeric compounds are partially, or even totally, removed by rinsing of the hair. Thus, the effect due to the polymeric compound is very limited after rinsing. Finally, that limits the effect of rinsed treatments (shampoo, conditioner) and also reduces the attractiveness of the compositions used in unrinsed form (lacquers, mousses, setting lotions . . . ), to the extent that the user loses the effect acquired through the treatment when he washes his hair.

It is possible to obtain an efficacious cosmetic effect, resistant to rinsing, without risking the problems of overladen hair in the case of cumulative effect and without being compelled to use organic solvents. There are used therefor organosiloxane compounds in the form of monomers soluble in water having at least one neutralizable chemical function, which is introduced into the water, and to which a pH-adjusting agent is added in proportions such that the organosiloxane compounds are partially but not totally neutralized.

In these cases, it is seen that the application of these compositions makes it possible to obtain marked cosmetic effects, with no problem in the event of cumulative effect. These effects are resistant to rinsing. Such compositions are described in the patents FR 2.783.164, FR 2.783.167 and FR 2.783.165.

Notwithstanding the attractive effects observed, this method has a major drawback: it concerns the pH of the product. In order to obtain the attractive effects, the pH of the formula must be in excess of 9, or even 10. It is known to the individual skilled in the art that the repeated application to the hair of products with a high pH can result in a weakening of the hair.

### DETAILED DESCRIPTION OF THE PREFERRED EMBODIMENTS

In a surprising and unexpected manner, the inventors have discovered that it is possible to respond to the above problems by using a mixture of an orthodiphenol and a catalytic system comprising a salt and/or an oxide of Mn(II) and/or Zn(II) and a hydrogenocarbonate of an alkaline metal.

In this application, there is understood by hairdressing effect the holding and/or setting of the hairstyle, this holding and/or setting of the hairstyle preferably being accompanied by, among others, one or more of the following properties: softness, pleasing feel, good untangling, smoothing, volume and body.

The invention specifically has as its preferred purpose a process for immediate densification of human keratinous fibers and more especially the hair, making it possible in particular to rapidly compensate for the loss of hair and to be coupled with a process for densification with a longer response time, such as those used to date or for dressing of the hair.

More precisely, the invention has as its preferred purpose a process for densification of human keratinous fibers comprising a stage which consists in applying to the keratinous fibers and the skin from which said fibers emerge and more especially to the hair and/or the scalp, a (cosmetic) composition containing, in a physiologically acceptable medium for topical application, an effective quantity of at least one precursor of densification, chosen from among the compounds comprising at least one aromatic cycle having at least two hydroxyl groups borne by two consecutive carbon atoms of the aromatic cycle, and an effective quantity of a catalytic system comprising a first constituent chosen from among the salts and oxides of Mn(II) and/or Zn(II) and mixtures thereof and a second constituent chosen from among the alkaline hydrogenocarbonates, the alkaline-earth hydrogenocarbonates and mixtures thereof, the proportions of the first and second constituents being such that:

[Mn(II)]/[HCO₃]≦1 with [Mn(II)]≠0

[Zn(II)]/[HCO₃]≦1 with [Zn(II)]≠0

[Mn(II)+Zn(II)]/[HCO₃]≦1 with [Mn(II) and [Zn(II)]≠0

In addition, this process may comprise a stage of contact of the precursor of densification and of the catalytic system with the keratinous fibers and/or the skin from which said fibers emerge, and in particular the hair and/or the scalp, and possibly a stage of rinsing of the fibers and skin adjacent to said fibers.

This treatment process has the characteristics of a cosmetic process to the extent that it makes it possible to improve the esthetics of the hair by giving it a greater strength and an enhanced appearance. In addition, it may be used daily for several months.

This process has the advantage of being rapid to implement, and of requiring an exposure time from 1 to 30 minutes, for example from 3 to 15 minutes. At the end of this exposure time, the hair density appears greatly increased.

The invention also relates to the use of the combination of at least one precursor of densification and a catalytic system such as defined previously, as an agent to increase the densification of the keratinous fibers, and in particular of the hair and/or to increase the density thereof.

The invention likewise relates to the (cosmetic) use of at least one precursor of densification and a catalytic system, such as defined previously, in a (cosmetic) composition for hair care for human beings, in order to increase the density of human keratinous fibers and in particular hair. It further has as its purpose the use of at least one precursor of densification and a catalytic system, such as defined previously, for the preparation of a hair composition for human beings, intended to increase the density of human keratinous fibers' such as the hair and the eyelashes or for the dressing of the hair.

In particular, the invention relates to the (cosmetic) use of the combination of at least one precursor of densification and a catalytic system, such as defined previously, in a cosmetic composition for hair care for human beings in order to treat androgenic alopecia or also to the use of the combination of at least one precursor of densification and a catalytic system, such as defined previously, for the preparation of a hair composition for human beings intended to treat androgenic alopecia. Thus, this composition makes it possible to maintain the hair in good condition and/or to compensate immediately for the natural loss of hair in men or it makes possible the dressing of the hair.

The catalytic chemical system present in the composition usable in the invention performs as a pseudo-oxidase capable of mimicking the oxidase activity without the drawbacks connected with the use of an enzymatic system. In particular, this composition for densification of keratinous fibers does not require the presence of an enzyme.

The effective quantity of precursor of densification and catalytic system corresponds to the quantity necessary for obtaining the desired result (that is, increasing the density of the hair). The individual skilled in the trade therefore is able to evaluate this effective quantity which depends on the nature of the precursor used, the nature of the catalytic system used, the individual on whom the precursor of densification and the catalytic system are applied, as well as the time of this application.

In the remainder of the text, and unless otherwise indicated, the quantities of the different ingredients of the composition are given in percentage by weight in relation to the total weight of the composition.

The composition usable in the invention therefore includes, in a physiologically acceptable medium, a) an effective quantity of at least one precursor of densification chosen from among the compounds comprising at least one aromatic cycle having at least two hydroxyl groups (OH) borne by two consecutive carbon atoms of the aromatic cycle and b) an effective quantity of a catalytic system comprising a first constituent chosen from among the salts and the oxides of Mn(II), the salts and the oxides of Zn(II), the dual salts and oxides of Mn(II) and Zn(II) and mixtures of these oxides and of these salts, and a second constituent chosen from among the alkaline hydrogenocarbonates, the alkaline-earth hydrogenocarbonates and mixtures thereof, the proportions of the first constituent and of the second constituents being such that:

[Mn(II)]/[HCO₃]≦1 with [Mn(II)]≠0

[Zn(II)]/[HCO₃]≦1 with [Zn(II)]≠0

[Mn(II)+Zn(II)]/[HCO₃]≦1 with [Mn(II) and [Zn(II)]≠0

where [Mn(II)], [Zn(II)] and [HCO₃] represent respectively the molar concentrations of Mn(II), Zn(II) and HCO₃ in the composition.

According to the invention, there may be used one or more precursors of densification combined with one or more salts and/or oxides of Mn(II), or with one or more salts and/or oxides of Zn(II), or with a mixture of salts and/or oxides of Mn((II) and of salts or oxides of Zn(II), or even with a mixture of dual salts and/or oxides of Mn(II) and Zn(II).

Generally, the ratio [Mn(II)]/[HCO₃] is chosen in the range from 10⁻⁵ to 10⁻¹. preferably from 10⁻³ to 10⁻² and typically is on the order of 5.10⁻³.

In the case where a salt or an oxide of Zn(II) is used alone, the ratio [Zn(II)]/[HCO₃] in general is on the order of 10 to 100 times higher than the ratio in the case of use of a salt or an oxide of Mn(II) alone.

Typically, this ratio is 10⁻⁴ or more, preferably 10⁻³ or more, and preferably on the order of 5.10⁻¹.

In the case of a mixture of Mn(II) and Zn(II), the ratio generally varies from 10-5 to 10-1, preferably 10⁻³ to 10-2, this ratio being chosen higher when the proportion of Zn(II) in the mixture is increased.

Generally, the molar concentration of Mn(II), Zn(II) or Mn(II)+Zn(II) in the final composition varies from 10⁻³ mM/I to 10 mM/I, preferably from 10⁻² mM/I to 1 mM/I.

When only one or more salts or oxides of Mn(II) is/are used, the molar concentration of Mn(II) in the final composition typically is from 10⁻³ mM/I to 10⁻¹ mM/I, and preferably from 10⁻² mM/I to 10⁻¹ mM/I.

In practice, when the catalytic system comprises only one or more salts or oxides of Zn(II), the concentration of Zn(II) in the final composition is from 5.10⁻² mM/I to 10 mM/I, better from 5.10⁻¹ mM/I to 1 mM/I.

Among the salts of Mn(II) and Zn(II) suitable for this invention, there may be cited chloride, fluoride, iodide, sulfate, phosphate, nitrate and perchlorate, the salts of carboxylic acids and mixtures thereof.

By way of example, there may be cited manganese chloride, manganese carbonate (for example rhodochrosite), Mn(II) difluoride, Mn(II) acetate tetrahydrate, Mn(II) lactate trihydrate, Mn(II) phosphate, Mn(II) iodide, Mn(II) nitrate trihydrate, Mn(II) bromide, Mn(II) perchlorate tetrahydrate and Mn(II) sulfate monohydrate.

The salts used advantageously are MnCl₂ and ZnCl₂.

The carboxylic acid salts usable in the invention also include the salts of hydroxylated carboxylic acids such as gluconate.

The salts of Mn(II) and Zn(II) may be introduced in solid form into the physiological medium or else derive from a natural water, mineral or thermal, rich in these ions (for example, in the form of hydroxidase) or also seawater (Dead Sea, in particular). They also may derive from clay (green clay, for example) or even from a plant extract containing them.

Among the alkaline and alkaline-earth hydrogenocarbonates, there may be cited the hydrogenocarbonates of Na, K, Mg, Ca and mixtures thereof, and in particular the hydrogenocarbonate of Na. These hydrogenocarbonates (also called bicarbonates) may derive from a natural water, for example spring water from the Vichy basin, from La Roche Posay, Badoit water.

As indicated previously, the catalytic chemical system used in accordance with the invention constitutes a specific pseudo-oxidase which makes possible the oxidation of orthodiphenols, in the presence of oxygen, as would be done by certain natural enzymatic catalysts having a polyphenoloxidase activity. In addition it makes possible, through oxidation of the precursor of densification, the coloring of keratinous fibers. Thus, gray hair can be colored brown, which enhances the perception of densification.

The precursors of densification in the compositions of the invention are compounds or mixtures of compounds including at least one aromatic cycle, preferably a benzene cycle, comprising at least two hydroxyl groups (OH) borne by two consecutive carbon atoms of the aromatic cycle.

The aromatic cycle may be a condensed aromatic cycle possibly containing one or more heteroatoms, such as naphthalene, tetrahydronaphthalene, indane, indene, anthracene, phenanthrene. indole, isoindole, indoline, isoindoline, benzofuran, dihydrobenzofuran, chromane, isochromane, chromene, isochromene, quinolein, tetrahydroquinolein and isoquinolein.

By way of example, the precursors of densification according to the invention may be represented by the following formula (I):

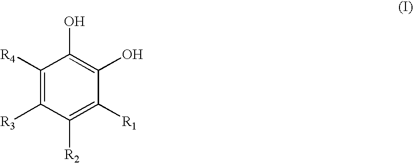

in which the subtituents R₁ to R₄, identical or different, represent a hydrogen atom, a halogen, hydroxyl, carboxyl, alkyl carboxylate, amino possibly substituted, linear or branched alkyl possibly substituted, linear or branched alkenyl possibly substituted, cycloalkyl possibly substituted, alcoxy, alcoxyalkyl, alcoxyaryl, the aryl group possibly able to be substituted, aryl, substituted aryl radical, an optionally substituted heterocyclic radical, a possibly substituted heterocyclic radical, a radical containing one or more silicon atoms, where two of the R₁ to R₄ substituents together form a saturated or unsaturated cycle possibly containing one or more heteroatoms and possibly condensed with one or more saturated or unsaturated cycles possibly containing one or more heteroatoms.

The saturated or unsaturated cycles, possibly condensed, also possibly may be substituted.

The alkyl radicals generally are C₁-C₁₀ alkyl radicals, preferably C₁-C₆ alkyl radicals, such as methyl, ethyl, propyl, butyl, pentyl and hexyl.

The alcoxy radicals in general are C₁-C₂₀ alcoxy radicals, such as methoxy, ethoxy, propoxy and butoxy.

The alcoxy alkyl radicals preferably are (C₁-C₂₀) alcoxy (C₁C₂₀) alkyl radicals, such as methoxymethyl, ethoxymethyl, methoxymethyl, ethoxyethyl, etc.

The cycloalkyl radicals in general are the C₄-C₈ cycloalkyl radicals, preferably cyclopentyl and cyclohexyl radicals. The cycloalkyl radicals may be cycloalkyl radicals substituted, in particular, with alkyl, alcoxy, carboxylic acid, hydroxyl, amine and ketone groups.

The alkenyl radicals preferably are C₂-C₂₀ radicals, such as ethylene, propylene, butylene, pentylene, methyl-2-propylene and decylene.

The radicals containing one or more silicon atoms preferably are polydimethylsiloxane, polydiphenylsiloxane, polydimethylphenylsiloxane, stearoxydimethicone.

The heterocyclic radicals in general are radicals comprising one or more heteroatoms chosen from among O, N and S, preferably O or N, possibly substituted with one or more alkyl, alcoxy, carboxylic acid, hydroxyl, amine or ketone groups.

Among the usable heterocyclic radicals, there may be cited the furyl, pyranyl, pyrrolyl, imidazolyl, pyrazolyl, pyridyl, thienyl groups.

Also preferably, the heterocyclic groups are condensed groups such as benzofuranyl, chromenyl xanthenyl, indolyl, isoindolyl, quinolyl, isoquinolyl, chromanyl, isochromanyl, indolinyl, isoindolinyl, coumarinyl, isocoumarinyl groups, these groups possibly able to be substituted, in particular with one or more OH groups.

The precursors of densification usable in the invention include in particular:

flavonols such as catechin and epicatechin gallate,

flavonols such as quercetin,

anthocyanidines such as peonidine,

anthocyanines, for example oenine,

hydroxybenzoates, for example the salts of gallic acid,

flavones such as luteolin,

iridoiles such as oleuropein, luteolin, oleuropein and the anthocyanines being able to be osylated (for example glucosylated) and/or in the form of oligomers (procyanidines),

hydroxystilbenes, for example tetrahydroxy-3,3′,4,5′-stilbene, possibly oxylated (for example glucosylated),

3,4-dihydroxyphenylalanine and derivatives thereof,

2,3-dihydroxyphenylalanine and derivatives thereof,

4,5-dihydroxyphenylalanine and derivatives thereof,

4,5-dihydroxyindole and derivatives thereof,

5,6-dihydroxyindole and derivatives thereof,

6,7 dihydroxyindole and derivatives thereof,

2,3-dihydroxyindole and derivatives thereof,

dihydroxycinnamates, such as caffeic acid and chlorogenic acid,

hydroxycoumarins,

hydroxyisocoumarins,

hydroxycoumarones,

hydroxyisocoumarones,

hydroxychalcones,

hydroxychromones,

anthocyanes,

quinones,

hydroxyxanthones,

1,2 dihydroxybenzenes,

1,2,4 trihydroxybenzenes,

1,2,3 trihydroxybenzenes,

2,4,5 trihydroxytoluene,

5,6 dihydroxyindoline, and

mixtures of the preceding compounds.

When the precursors of densification have D and L forms, the two forms may be used in the compositions according to the invention, as well as the racemics.

The quantity of precursors of densification in the final composition is preferably sufficient to obtain a densification visible to the naked eye. This quantity may vary to a large extent in terms of the nature of the precursor and the intensity desired for the densification.

In general, a suitable densification is obtained when the precursor or precursors of densification represents/represent at least 0.001% by weight of the total weight of the composition. It/they may represent up to 80% of the total weight of the composition. In practice, it/they represents/represent from 0.01% to 25% and better from 2% to 8% of the total weight of the composition.

By varying the nature of the different precursors of densification and the proportions thereof in the composition, the densification as well as the color of the keratinous fibers may be varied, after application of the composition to said fibers.

For example, a 1/10 ratio of chlorogenic acid and catechin may be used and a brown coloring of the hair obtained, or a 1/1 ratio and a mahogany coloring obtained.

The precursors of densification may be extracts of plants, fruits, citrus fruits, vegetables, trees, bushes and mixtures of these extracts, which contain numerous polyphenols such as previously defined.

Among the plant extracts, there may be cited extracts of rose, sorghum and tea.

Among the fruit extracts, there may be cited extracts of apple, grape (in particular grape seeds), cacao (beans and/or pods) and banana.

Among the vegetable extracts, there may be cited the extract of potato.

Among the tree extracts, there may be cited pine bark.

Mixtures of plant and/or fruit extracts such as mixtures of potato and tea extracts and mixtures of grape and apple extracts also may be used.

The densification obtained is different according to the portions of fruits used, for example grape pulp or seeds.

In particular, the precursor of densification is chosen from among the extracts of tea, grape, apple, banana, cacao, sorghum, potato and mixtures thereof.

The composition of the invention may be for cosmetic or pharmaceutical use. Preferentially, the composition of the invention is for cosmetic use. Also, the composition must contain a nontoxic physiologically acceptable medium capable of being applied to the skin and the phaneric structures of human beings. By “cosmetic” there is understood, in the meaning of the invention, a composition with pleasing appearance, scent and feel.

The physiologically acceptable medium is a solid or liquid medium not impairing the densification property of the precursors or the catalytic effect of the catalytic system. Advantageously, it facilitates “in situ” polymerization of the orthodiphenols, which imparts thereto a bacteriostatic effect.

This composition may be in any known galenical forms suited to a direct application to the scalp and/or to the hair (of the topical application type).

For a topical application to the skin, the composition may have the form of an aqueous, alcoholic or hydro-alcoholic solution or suspension or an oily suspension, an emulsion with a more or less fluid consistency and in particular liquid or semi-liquid, obtained by dispersion of a fatty phase in an aqueous phase (oil/water) or inversely (water/oil), a solid (oil/water) or (water/oil) emulsion, an aqueous, hydro-alcoholic or oily gel more or less fluid or solid, a loose or compacted powder to be used as is or to be incorporated into a physiologically acceptable medium, or also microcapsules or microparticles, vesicular dispersions of ionic and/or nonionic type. It thus may be in the form of a lotion, serum, milk, oil/water or water/oil cream, ointment, pomade, balm, patch, moistened pad.

A composition in the form of mousse or also in the form of an aerosol or spray, then including a propellent under pressure, likewise may be contemplated.

In particular, the composition for application to the scalp or the hair may be in the form of a lotion for hair care, for example for daily or biweekly application, a shampoo or a hair conditioner, in particular for biweekly or weekly application, a liquid or solid soap for cleansing of the scalp for daily application, a hairstyling product (lacquer, setting product, hairdressing gel), a treating pack, a lathering cream or gel for cleansing of the hair. It also may be in the form of a hair dye or mascara to be applied with a brush or a comb.

According to a specific embodiment, the composition according to the invention is in the form of hair cream or lotion, shampoo or hair conditioner.

The quantities of the different constituents of the physiological medium of the composition according to the invention are those generally used in the areas considered. In addition, these compositions are prepared in accordance with the usual methods.

When the composition is an emulsion, the proportion of the fatty phase may range from 2% to 80% by weight, and preferably from 5% to 50% by weight in relation to the total weight of the composition. The aqueous phase is adjusted in terms of the content of fatty phase and of compound(s) (I) as well as that of possible additional ingredients, in order to obtain 100% by weight. In practice, the aqueous phase represents from 5 to 99.9% by weight.

The fatty phase may contain fatty or oily compounds, liquid at room temperature (25° C.) and atmospheric pressure (760 mmHg), generally referred to as oils. These oils may be compatible or incompatible with each other and form a macroscopically homogeneous liquid fatty phase or a bi- or triphase system.

In addition to oils, the fatty phase may contain waxes, gums, lipophilic polymers, “pasty” or viscous products containing solid portions and liquid portions.

The physiologically acceptable medium preferably is a medium comprising a phase making the precursors of densification but also the catalytic system soluble. According to the invention, by making soluble there is understood that at least 0.005% of each of the constituents of the invention are dissolved in the medium.

Among the solvents for precursors of densification suitable for formulation of the compositions usable in the invention, there may be cited water, mono- or dihydroxylated aliphatic alcohols and the polyols, ethers of polyols and mixtures thereof. The alcohols may be mono- or dihydroxylated aliphatic alcohols. Aromatic alcohols also may be used.

Among these solvents, there may be cited by way of example the lower C₁-C₈ and better C₁-C₄ alkanols with one hydroxyl group such as ethanol and isopropanol; the alkanediols such as ethylene glycol or butanediol-1,4, propylene glycol; the polyols or ethers of glycols, such as 2 butoxyethanol, glycerin, sorbitol, diglycerin, monoethylether and monomethylether of diethylene glycol; acetone as well as the aromatic alcohols such as benzyl alcohol or phenoxyethanol and analogue products such as 1-phenoxy-2-propanol or mixtures thereof.

The solvents preferably are present in proportions ranging from 1 to 80% by weight, and in particular from 5 to 60% by weight in relation to the total weight of the composition, for example from 10% to 30% by weight.

The alcohols advantageously are chosen from among in particular the lower (C₁-C₄) alkanols, such as ethanol and isopropanol and in particular the C₂-C₆ alkanediols such as propylene glycol and pentane diol.

The physiologically acceptable medium preferably comprises water (in particular distilled or exchanged) or a water/alcohol mixture, in particular water/ethanol.

The quantity of alcohol in the water/alcohol mixture may represent up to 80% by weight of the water/alcohol mixture, preferably from 1 to 50% by weight and better 5 to 20% by weight.

In addition, the composition usable in the invention may contain at least one surfactant agent representing at least 0.01% of the total weight of the composition. In practice it represents from 0.1% to 40% of the total weight of the composition, for example from 0.5 to 30% by weight and better from 10% to 20% by weight.

The surfactant agents may be chosen from among the anionic, cationic, nonionic, amphoteric surfactant agents or mixtures thereof, and preferably from among the nonionic surfactants. These surfactant agents are in particular those used in shampoos and conditioners for human hair, having a cleansing effect.

Among these surfactant agents, there may be cited alkylbenzene sulfonates, alkylnaphthalene sulfonates, sulfates, sulfate ethers and the sulfonates of fatty alcohols (with at least 12 carbon atoms), quaternary ammonium salts, such as trimethylcetyl ammonium bromide or cetylpyridinium bromide, ethanolamides of fatty acids possibly oxyethylene, polyoxyethylene acids, alcohols or amines, polyglycerol alcohols, polyoxyethylene alkylphenols or polyglycerols, as well as polyoxyethylene alkylsulfates.

When the composition according to the invention is in the form of an emulsion, it contains in particular one or more emulsifiers and/or co-emulsifiers such as those generally used in the cosmetic and pharmaceutical areas. In addition, their nature is a function of the significance of the emulsion. In practice, the emulsifier and possibly the co-emulsifier are present in the composition in a proportion ranging from 0.1% to 30% by weight, preferably from 0.5 to 20% by weight and better from 1 to 8%. In addition, the emulsion may contain lipid vesicles and in particular liposomes.

As emulsifiers usable in the invention, there may be cited for example stearate or laurate of glycerol, the stearates or oleates of sorbitol, alkyl dimethiconecopolyols (with ≧8 alkyl) and mixtures thereof for a water/oil emulsion. There also may be used the monostearate or monolaurate of polyethylene glycol, the stearate or oleate or polyoxyethylene sorbitol, dimethiconecopolyols and mixtures thereof for an oil/water emulsion.

The composition of the invention further may comprise other ingredients usually used in the areas concerned, chosen from among the thickeners or gelling agents with a hydro-alcoholic phase or an oily phase, coloring agents soluble in the medium of the composition, solid particles of the filler or pigment type, antioxidants, preservatives, fragrances, electrolytes, neutralizers, U.V. blocking agents such as sunscreens, cosmetic and pharmaceutical active principles, mixtures thereof. These additional ingredients may be present in the composition in accordance with the quantities generally used in the cosmetic and dermatological area and in particular in the ratio of 0.01 to 50% of the total weight of the composition and better from 0.1 to 20% and for example from 0.1 to 10%.

As hydro-alcoholic gelling agents or thickeners usable in the invention, there may be cited carboxyvinyl polymers (carbomer), acrylic copolymer such as the acrylate/alkylacrylate copolymers and the acrylic acid polymers, polyacrylamides, polysaccharides such as hydroxypropylcellulose or hydroxyethylcellulose, natural gums and clays such as gum arabic, xanthane gum, sodium alginate, mixtures thereof and, as lipophilic gelling agents or thickeners, there may be cited modified clays such as the Bentones, metallic salts of fatty acids such as aluminum stearates, hydrophobic treated silica, ethylcellulose, mixtures thereof.

In practice, these gelling agents or thickeners are present in proportions ranging from 0.1 to 5% and in particular from 0.2 to 3% by weight in relation of the total weight of the composition.

The individual skilled in the trade, of course, will take care to choose the previously indicated possible additional ingredient or ingredients in such manner that the advantageous properties intrinsically attached to the composition for densification according to the invention are not, or are not substantially, impaired by the contemplated addition or additions. In particular, the antioxidants and preservatives present in the composition are such that they must not interfere with the densification reaction, and in particular not react with the salts and oxides of Mn(II) or Zn(II), the hydrogenocarbonates and the precursors of densification, such as the polyphenols having a repetition moiety of the orthophenol type.

The composition according to the invention may contain one or more agents for chelation of the ions of the catalytic system, on condition that these agents be in a quantity such that the Mn(II) and/or Zn(II) ions subsist in the composition in sufficient quantity to catalyze the oxidation of the densification compound. In other words, the molar quantity of chelation agent must be lower than that of the Mn(II) and/or Zn(II) ions present in the composition.

The composition according to the invention advantageously is free of agents for chelation of the Mn(II) and/or Zn(II) salts used, because these agents tend to inhibit oxidation of the precursors of densification.

The composition usable in the invention has in particular a pH which ranges from 7 to 12, preferably from 7 to 9, and typically on the order of 8. This pH, close to neutrality, makes it possible to use the densification process according to the invention on individuals with a sensitive scalp. This pH is adjusted through the use of alkalizing or acidifying agents well known in the state of the art for the treatment of human keratinous fibers.

Among the alkalizing agents, there may be cited by way of example ammonia, alkaline carbonates, alkanolamines such as the mono-, di- and triethanolamines as well as derivatives thereof, sodium or potassium hydroxides and the compounds of the following formula (V):

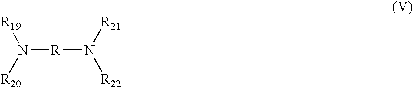

in which:

R is a propylene remainder possibly substituted with a hydroxyl group or a C₁-C₄ alkyl radical; and

R₁₉, R₂₀, R₂, and R₂₂. identical or different, represent a hydrogen atom, a C₁-C₄ alkyl or C₁-C₄ hydroxyalkyl radical.

The acidifying agents usable in the invention in the case, for example, of a composition with two compartments and in order to stabilize the orthodiphenol+Mn(II) and/or Zn(II) mixture, the quantity of bicarbonate in the second compartment will be adjusted to have a pH in excess of 7, at the time of application. By way of example, there may be used organic or inorganic acids such as hydrochloric acid, orthophosphoric acid, carboxylic acids such as citric acid, lactic acid, or the sulfonic acids.

The densifying effect may be improved by using organic or inorganic acids such as described previously after the catalytic reaction took place on the scalp. The persistence of the effect (in particular the staying power of the densification after several shampoos) is increased by the setting properties of the acids. Furthermore, the modification of the pH, following the action of an acid, can modify the densifying effect and possibly weaken it. In this case, the application of an organic or inorganic base will increase the intensity of the densifying effect. These adjustments in the densification effect make it possible to adapt this effect in terms of the desire of the user.

As oils usable in the invention, there may be cited oils of mineral origin (vaseline oil, hydrogenated isoparaffin), oils of vegetable origin (liquid fraction of shea butter, sunflower, apricot, alcohol or fatty acid oil), oils of animal origin (perhydrosqualene), synthetic oils (ester of fatty acid, Purcellin oil), silicone oils (linear or cyclic polydimethylsiloxane, phenyltrimethicone) and fluorous oils (perfluoropolyethers). As waxes, there may be cited silicone waxes, beeswax, carnauba or paraffin waxes, polyethylene waxes.

In addition, the composition may contain an additional cosmetic or dermatological active principle which may be hydrophilic and chosen from among the proteins or protein hydrolysates, amino acids, polyols, urea, allantoin, sugars and sugar derivatives, hydrosoluble vitamins, plant extracts other than those previously described (for example those of iridaceous plants or soybean), ferric or ferrous salts and the hydroxy acids; or lipophilic and chosen from among retinol (vitamin A) and derivatives thereof, in particular ester (retinol palmitate), (tocopherol (vitamin E) and derivatives thereof, in particular ester tocopherol acetate), essential fatty acids, ceramides, essential oils, salicylic acid and derivatives thereof such as n-octanoyl-5 salicylic, the esters of hydroxy acids, phospholipids such as lecithin, mixtures thereof.

The composition according to the invention makes possible an immediate densification of the keratinous fibers, and in particular of the hair. Thus, after 3 minutes of topical application of the composition according to the invention to the fibers and the skin from which the fibers emerge, and more especially to the hair and the scalp, a densification effect is observed by the naked eye, an effect which reaches its full magnitude in 8-15 minutes following application, or even at the end of 12-72 hours depending on the physiological medium used.

According to a particular embodiment of the invention, the action of immediate densification of the precursor of densification+catalytic system combination may be coupled with the action of additional compounds increasing hair density and/or promoting regrowth of the keratinous fibers, in particular the hair, and/or limiting the loss of these fibers (hair in particular). These additional active compounds may have slow or delayed action, with a view to being substituted over time for the combination according to the invention with immediate effect. These additional active compounds are those known to the individual skilled in the trade to have an effect on hair density.

Advantageously, these active compounds with a delayed effect, increasing hair density and/or promoting regrowth of the keratinous fibers and/or limiting loss thereof, hereinafter referred to as delayed effect hair restoration/regrowth active principles, are active principles for topical application, applied locally to the fibers (hair in particular) and/or the skin from which these fibers emerge (scalp in particular).

Likewise, the invention also has as its purpose a composition for care of the human keratinous fibers and/or the skin from which these fibers emerge, and more especially a composition for care of the hair and/or of the scalp, containing in a physiologically acceptable medium, a) an effective quantity of at least one precursor of densification chosen from among the compounds comprising at least one aromatic cycle having at least two hydroxyl groups (OH) borne by two consecutive carbon atoms of the aromatic cycle, b) an effective quantity of a catalytic system comprising a first constituent chosen from among the salts and oxides of Mn(II) and/or Zn(II) and mixtures thereof and a second constituent chosen from among the alkaline hydrogenocarbonates, the alkaline-earth hydrogenocarbonates and mixtures thereof, and c) an effective quantity of at least one active principle with delayed effect increasing hair density and/or promoting regrowth of the keratinous fibers, in particular the hair, and/or limiting the loss of these fibers, and in particular of the hair, the proportions of the first constituent and of the second constituents being such that:

[Mn(II)]/[HCO₃]≦1 with [Mn(II)]≠0

[Zn(II)]/[HCO₃]≦1 with [Zn(II)]≠0

[MN(II)+Zn(II)]/[HCO₃]≦1 with [Mn(II)] and [Zn(II)]≠0

where [Mn(II)], [Zn(II)] and [HCO₃] represent respectively the molar concentrations of Mn(II). Zn(II) and HCO₃ in the composition.

The additional active compounds with delayed effect advantageously are chosen in particular from among the lipooxygenase inhibitors such as described in EP 0648488 and in particular the derivatives of catecholic butanes such as nordihydroguaiaretic acid (NDGA) and its enantiomer masoprocol, the bradykinin inhibitors described in particular in EP 0845700, the prostaglandins and derivatives thereof, in particular those described in WO 98/33497, WO 95/11003, JP 97-100091, JP 96-134242, the agonists or antagonists of the prostaglandin receptors, the non-prostanoic analogues of prostaglandins such as described in EP 1175891 and EP 1175890, WO 01/74307, WO 01/74313, WO 01/74314, WO 01/74315 or WO 01/72268, mixtures thereof.

As other additional compounds promoting the growth of hair which may be present in the composition according to the invention, there may be cited vasodilators, antiandrogens, cyclosporins and analogues thereof, antimicrobial and antifungal agents, anti-inflammatory agents, retinoids, alone or in a mixture.

The vasodilators usable are in particular potassium channel agonists including minoxidil, as well as the compounds described in the U.S. Pat. Nos. 3,382,247, 5,756,092, 5772990, 5760043, 5466694, 5438058, 4973474, cromakalim, nicorandil and diaxozide, alone or in combination.

The antiandrogens include in particular the steroidal or nonsteroidal inhibitors of 5α-reductase, such as finasteride and the compounds described in U.S. Pat. No. 5,516,779, cyprosterone acetate, azelaic acid, the salts and derivatives thereof and the compounds described in U.S. Pat. No. 5,480,913, flutamide and the compounds described in the U.S. Pat. Nos. 5,411,981, 5,565,467 and 4910226.

The antimicrobial or antifungal compounds may be chosen from among the derivatives of selenium, ketoconazole, octopirox, triclocarban, triclosan, zinc pyrithione, itraconazole, asiatic acid, hinokitiol, mipirocine, tetracyclines, in particular erythromycin and the compounds described in EP 0680745, clinycin hydrochlorate, benzoyl or benzyl peroxide and minocycline.

The anti-inflammatory agents may be chosen from among the steroidal anti-inflammatory agents such as the glucocorticoids, the corticosteroids (for example: hydrocortisone), and the nonsteroidal anti-inflammatory agents such as glycyrrhetinic acid and α-bisabolol, benzydamine, salicylic acid and the compounds described in EP 0770399, WO 94/06434 and FR 2268523.

The retinoids may be chosen from among isotretinoin, acitretin and tazarotene.

As other active compounds with delayed effect to promote the growth and/or to limit the loss of hair usable in combinations with the precursor of densification and the catalytic system previously described, there may be cited aminexil, 6-0-[(9Z, 12Z)-octadeca-9,1 2-dienoyl]hexapyranose, benzalkonium chloride, benzethonium chloride, phenol, estradiol, chlorpheniramine maleate, the derivatives of chlorophylline, cholesterol, cysteine, methionine, menthol, peppermint oil, calcium pantothenate, panthenol, resorcinol, C protein kinase activators, glycosidase inhibitors, glycosaminoglycanase inhibitors, esters of pyroglutamic acid, hexosaccharidic or acyl-hexosaccharidic acids, substituted aryl ethylenes, N-acylated amino acids, flavonoids, derivatives and analogues of ascomycin, histamine antagonists, saponines, proteoglycanase inhibitors, estrogen agonists and antagonists, pseudoterins, cytokins and growth-factor promoters, IL-1 or IL-6 inhibitors, IL-10 promoters, TNF inhibitors, hydroxy acids, benzophenones and hydantoin, retinoic acid; vitamins such as vitamin D, analogues of vitamin B12 and pantothenol; triterpenes such as ursolic acid and the compounds described in U.S. Pat. No. 5,529,769, U.S. Pat. No. 5,468,888, U.S. Pat. No. 5,631,282; antipruriginous agents such as thenaldine, trimeprazine or cyproheptadine; antiparasitics, in particular metronidazole, crotamiton or the pyrethrinoids; antifungal agents, in particular octopirox and the compounds belonging to the class of imidazoles such as econazole, ketoconazole or miconazole or salts thereof, esters of nicotinic acid, including in particular tocopherol nicotinate, benzyl nicotinate and the C₁-C₆ alkyl nicotinates such as the methyl or hexyl nicotinates; the NO donors such as sodium dihydrate nitroprussiate or nitrosoglutathione; guanylate-cyclase inhibitors such as sildenafil (Viagra®), calcium antagonist agents, such as cinnarizine, diltiazem, nimodipine, verapamil, alverine and nifedipine; hormones such as estriol or analogues thereof, thyroxin and salts thereof, progesterone; antiandrogenic agents such as oxendolone, spironolactone, diethylstilbestrol and flutamide; metalloproteinase inhibitors (MMP9) such as described in FR 2778558; mixtures thereof.

In particular, the active principle with delayed effect is chosen from among the lipoxygenase inhibitors, and in particular from among the derivatives of catecholic butanes such as nordihydroguaiaretic acid (NDGA) and its enantiomer masoprocol, and vasodilators and in particular from among the potassium channel agonists including minoxidil, combinations thereof.

The effective quantity of an active principle with delayed effect corresponds to the quantity necessary to obtain the desired result (that is, increasing the density of the hair). The individual skilled in the trade therefore is able to evaluate this effective quantity which depends on the nature of the compound used, the individual to whom it is applied, and the time of this application.

To provide an order of magnitude, according to the invention, the active hair restoration/regrowth compound with delayed effect can be used, for topical application, in a quantity representing from 10⁻⁴% to 8% of the total weight of the composition and better in a quantity representing from 10⁻²% to 5% of the total weight of the composition, for example from 0.5 to 3%.

It also is possible according to the invention to couple the precursor of densification+catalytic system combination with hair restoration/regrowth active principles used orally, such as finasteride and/or dietary supplements, in particular iron and/or amino acids such as cystine and taurine. In practice, the quantity of hair restoration/regrowth active principles used orally may represent from 0.1 to 300 mg per day, for example from 5 to 10 mg/day.

The process of densification according to the invention may be applied to human keratinous fibers such as the hair, eyelashes, eyebrows and beard and more especially to the hair.

For the densification of keratinous fibers, different processes of application according to the invention may be used.

According to a first process, there is applied on the keratinous fibers, in the presence of oxygen, for example from the air, a composition comprising all the ingredients of the composition of the invention.

According to a second process, there initially may be applied to the keratinous fibers a first composition of one or more precursors of densification in a physiologically acceptable medium, then to this first composition, a second composition containing the catalytic system in a physiologically acceptable medium. Obviously, the order of application of these compositions may be reversed.

The application of the compositions may be performed by any known means, in particular by spraying.

The compositions according to the invention may be offered and packaged in different forms.

According to a first embodiment, the composition usable for the invention may be packaged in the form of an aerosol with a single compartment in which is found the composition containing the precursor or precursors of densification, the catalytic system and a conventional inert propellant such as nitrogen, a saturated hydrocarbon such as isopropane or a halogenated hydrocarbon.

In a second embodiment, the composition usable for the implementation of the invention may be packaged in the form of a kit comprising two distinct containers, one for the base composition containing the precursor or precursors of densification, the other for the catalytic system, the base composition and the catalytic system being mixed or applied successively at the time of use.

In a third embodiment, the composition may be contained in a pump system with a single compartment, without air uptake, or in a pump system with two compartments, the precursor of densification being in one compartment and the catalytic system in the other compartment.

In a fourth embodiment, the composition contains in a first compartment a first solution of salt(s) and/or oxide(s) of Mn(II) and/or Zn(II) and of one or more orthodiphenols, and in another compartment a second solution containing the bicarbonate; the active principle for hair restoration/regrowth with delayed effect being present in one or the other of the compartments.

The composition according to the invention may be applied to the balding areas of the scalp and the hair of an individual, and possibly left in contact for several hours and possibly rinsed off.

For example, one may apply the composition containing an effective quantity of a compound of formula (I), salified or unsalified, in the evening, leave it in contact throughout the night and possibly shampoo in the morning. These applications may be repeated daily for one or more months according to the individuals.

There now will be presented, by way of illustration, examples of implementation of the invention which in no way would limit its scope.

### EXAMPLE 1

### Lotion for Immediate Densification

| | Quantity and method |
|---|---|
| Compositions | of use |
| Propylene Glycol/ethanol/H₂O (33/33/qsp 100) | Solution 1* to be applied |
| Mn 1 mM (55 mg/l), | to hair and scalp |
| Propyl gallate 3%/NDGA** 1%/catechin 0.5% | |
| Sodium bicarbonate 1 M in H₂O | Solution 2* |
| | (densification developer) |

**NDGA represents nordihydroguaiaretic acid.

Solutions 1 and 2 were applied successively to a balding scalp with hair of a natural “salt and pepper” color. The formulations were allowed to act on the hair for one hour, then were rinsed off with warm tap water.

As early as 3 minutes after application, a densification effect, which reaches its full magnitude in 8-15 minutes after application, is perceptible to the naked eye.

This is apparent from the attached photos. The photo on the left corresponds to time t=0, just prior to application of solution 1, then 2, and the photo on the right corresponds to t=8 min, after application.

### EXAMPLE 2

### Cosmetic Lotion for Immediate Densification Incorporating an Active Principle for Hair Restoration, with Delayed Effect

| | Dosage and |
|---|---|
| Compositions | method of use |
| Propylene Glycol/ethanol/H₂O (33/33/qsp 100) | Solution 1 to be |
| | applied to hair and scalp |
| Mn 1 mM (55 mg/l), | |
| Propyl gallate 3%/NDGA 1%/catechin 0.5% | |
| Aminexil 1.5% | |
| Sodium bicarbonate 1 M in | Solution 2 |
| H₂O (exchanged) | (densification developer) |

### EXAMPLE 3

### Pharmaceutical Lotion for Immediate Densification Incorporating an Active Principle Stimulating Regrowth of the Hair with Delayed Effect

| | Dosage and |
|---|---|
| Compositions | method of use |
| Propylene Glycol/ethanol/H₂O (33/33/qsp 100) | Solution 1 to be applied |
| | to hair and scalp |
| Mn 1 mM (55 mg/l), | |
| Propyl gallate 3%/NDGA 1% catechin 0.5% | |
| Minoxidil 2% | |
| Sodium bicarbonate | Solution 2 |
| 1 M in H₂O | (densification developer) |

The compositions of examples 2 and 3 also were tested. They thus were applied successively to white and pigmented hair as well as to the scalp. Next they were allowed to act on the hair for one hour, then the treated hair was rinsed with warm tap water. Out of 9 individuals having tested these compositions, 5 had an immediate impression of redensification and 4 had a slight impression of immediate redensification.

The combination of orthodiphenols with the catalytic system imparts a good densification to the hair. Moreover, this densification is resistant to several shampoos.

Finally, the pH close to neutrality for these formulations makes it possible to contemplate a use of the process of the invention even for the hair of individuals having a sensitive scalp.

By varying the quantity of orthodiphenols used and the type of molecule used, the densification may be adjusted in terms of the phototype (in particular color of the skin and hair) of the consumer.

### EXAMPLE 4

### Hairdressing

The following compositions are prepared by simple mixing (protected from the air for composition 1):

COMPOSITIONS

| | 1 | | A (comparative) | |
|---|---|---|---|---|
| L-DOPA | 1 | g | — | |
| MnCl₂ | 0.2 | g | 0.2 | g |
| NaHCO₃ | 1 | g | 1 | g |
| Water | 50 | ml | 50 | ml |
| Ethanol | 50 | ml | 50 | ml |

The two compositions are introduced into a container which is equipped with a spraying system of the pump-bottle type.

Application and Evaluation:

Two wigs with 15 g of natural hair are prepared. The hair is held at the roots on a rubber band and left free over the remainder of its length.

The compositions were sprayed onto the wigs (2 g per wig). The wigs were left in this state until they dried.

The cosmetic properties, and in particular the hairdressing effect, are noted. Then the hair is washed with a sodium sulfate laurylether shampoo, then dried again. The cosmetic properties are noted for a second time.

| | Hairdressing | Hairdressing |
|---|---|---|
| Composition | effect before washing | effect after washing |
| 1 | 30 | 25 |
| A | 30 | 0 |

The results show that the application to the hair of a composition according to the invention imparts thereto a hairdressing effect resistant to washing.

The above written description of the invention provides a manner and process of making and using it such that any person skilled in this art is enabled to make and use the same, this enablement being provided in particular for the subject matter of the appended claims and including the use of an effective quantity of at least one precursor of densification, chosen from among the compounds comprising at least one aromatic cycle having at least two hydroxyl groups borne by two consecutive carbon atoms of the aromatic cycle, and an effective quantity of a catalytic system comprising a first constituent chosen from among the salts and oxides of Mn(II) and/or Zn(II) and mixtures thereof and a second constituent chosen from among the alkaline hydrogenocarbonates, the alkaline-earth hydrogenocarbonates and mixtures thereof, in a physiologically acceptable medium for topical application, as an agent for increasing the density of human keratinous fibers and in particular the hair or for dressing of the hair, the proportions of the first and second constituents being such that:

[Mn(II)]/[HCO₃]≦1 with [Mn(II)]≠0

[ZN(II)]/[HCO₃]≦1 with [ZN(II)]≠0

[Mn(II)+Zn(II)]/[HCO₃]≦1 with [MN(II)] and Zn(II)]≠0

where [Mn(II)], [Zn(II)] and [HCO₃] represent respectively the molar concentrations of Mn(II), Zn(II) and HCO₃ in the composition. Also fully described and enabled is the (cosmetic) use of an effective quantity of at least one precursor of densification, chosen from among the compounds comprising at least one aromatic cycle having at least two hydroxyl groups borne by two consecutive carbon atoms of the aromatic cycle, and an effective quantity of a catalytic system comprising a first constituent chosen from among the salts and oxides of Mn(II) and/or Zn(II) and mixtures thereof and a second constituent chosen from among the alkaline hydrogenocarbonates, the alkaline-earth hydrogenocarbonates and mixtures thereof, in a cosmetic composition for hair care for human beings in order to increase the density of the hair and/or to treat androgenic alopecia or for dressing of the hair. Similarly fully described and enabled is the use of an effective quantity of at least one precursor of densification, chosen from among the compounds comprising at least one aromatic cycle having at least two hydroxyl groups borne by two consecutive carbon atoms of the aromatic cycle, and an effective quantity of a catalytic system comprising a first constituent chosen from among the salts and oxides of Mn(II) and/or Zn(II) and mixtures thereof and a second constituent chosen from among the alkaline hydrogenocarbonates, the alkaline-earth hydrogenocarbonates and mixtures thereof, for the manufacture of a hair composition for human beings, intended to increase the density of the hair and/or to treat androgenic alopecia or for dressing of the hair.

All references, texts, documents, tests, applications, patents, publications, standards, etc. mentioned herein are incorporated herein by reference. Where a numerials range or limit is mentioned, all values and sub-ranges therewithin are specifically included as if written out. Where a term or terms appear in parentheses they are optional, unless descriptive of a preceding word or phrase. For example, a (cosmetic) composition is any composition, including a cosmetic composition.

1. A composition for care of the human keratinous fibers and/or the skin from which these fibers emerge, comprising, in a physiologically acceptable medium, a) an effective quantity of at least one precursor of densification chosen from among the compounds comprising at least one aromatic cycle having at least two hydroxyl groups (OH) borne by two consecutive atoms of the aromatic cycle, b) an effective quantity of a catalytic system comprising a first constituent chosen from among the salts and oxides of Mn(II) and/or Zn(II) and mixtures thereof and a second constituent chosen from among the alkaline hydrogenocarbonates, the alkaline-earth hydrogenocarbonates and mixtures thereof, and c) an effective quantity of at least one active principle with delayed effect increasing hair density and/or promoting regrowth of the keratinous fibers, in particular of the hair, and/or limiting the loss of these fibers and in particular of the hair, the proportions of the first constituent and the second constituent being such that:
[Mn(II)]/[HCO₃]≦1 with [Mn(II)]≠0
[Zn(II)]/[HCO₃]≦1 with [Zn(II)]≠0
[Mn(II)+Zn(II)]/[HCO₃]≦1 with [Mn(II)] and [Zn(II)]≠0
where [Mn(II)], [Zn(II)] and [HCO₃] represent respectively the molar concentrations of Mn(II), Zn(II) and HCO₃ in the composition.

2. The composition according to claim 1, wherein the ratio [Mn(II)]/[HCO₃] is chosen in the range from 10⁻⁵ to 10⁻¹.

3. The composition according to claim 1, wherein the ratio [Mn(II)]/[HCO₃] is chosen in the range from 10⁻³ to 10⁻².

4. The composition according to claim 1, wherein the ratio [Zn(II)]/[HCO₃] is chosen in the range from 10⁻⁴ to <1.

5. The composition according to claim 1, wherein the ratio [Zn(II)]/[HCO₃] is chosen in the range from 10⁻³ to <1.

6. The composition according to claim 1, wherein the ratio [Mn(II)+Zn(II)]/[HCO₃] is chosen in the range from 10⁻⁵ to 10⁻¹, preferably 10⁻³ to 10^{-2.}

7. The composition according to claim 1, wherein the salts of Mn(II) and Zn(II) are chosen from among chloride, fluoride, iodide, sulfate, phosphate, nitrate, perchlorate, the salts of carboxylic acids and mixtures thereof.

8. The composition according to claim 1, wherein the salt of Mn(II) and/or Zn(II) is chloride.

9. The composition according to claim 7, wherein the salts of carboxylic acids are salts of hydroxylated carboxylic acids.

10. The composition according to claim 7, wherein the salt of hydroxylated carboxylic acid is gluconate.

11. The composition according to claim 1, wherein the hydrogenocarbonate is chosen from among sodium hydrogenocarbonate, potassium hydrogenocarbonate, magnesium hydrogenocarbonate, calcium hydrogenocarbonate and mixtures thereof.

12. The composition according to claim 1, wherein the aromatic cycle comprising at least two hydroxy groups on two consecutive carbon atoms of the precursor of densification is a benzene cycle or a condensed aromatic cycle.

13. The composition according to claim 1, wherein the precursor of densification is a compound of formula (I): 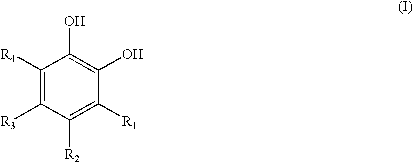
in which R₁ to R₄, identical or different, represent a hydrogen atom, a halogen, an hydroxyl, carboxyl, alkyl carboxylate, amino possibly substituted, linear or branched alkyl possibly substituted, linear or branched alkenyl possibly substituted, cycloalkyl possibly substituted, alcoxy, alcoxyalkyl, alcoxyaryl, the aryl group possibly able to be substituted, aryl, substituted aryl radical, a possibly substituted heterocyclic radical, a radical possibly containing at least one silicon atom, or two of the R₁ to R₄ constituents together may form a saturated or unsaturated cycle possibly containing at least one heteroatom and possibly condensed with at least one saturated or unsaturated cycle possibly containing at least one heteroatom.

14. The composition according to claim 1, wherein the precursor of densification is chosen from among flavonols, flavonols, anthocyanidines, anthocyanines, hydroxybenzoates, flavones, iridoids, the anthocyanines possibly being able to be osylated and/or in the form of oligomers, hydroxystilbenes possibly osylated, 3,4-dihydroxyphenylalanine and derivatives thereof, 2,3-dihydroxyphenylalanine and derivatives thereof, 4,5-dihydroxyphenylalanine and derivatives thereof, 4,5-dihydroxyindole and derivatives thereof, 5,6-dihydroxyindole and derivatives thereof, 6,7-dihydroxyindole and derivatives thereof, 2,3-dihydroxyindole and derivatives thereof, dihydroxycinnamates, hydroxycoumarines, hydroxyisocoumarines, hydroxycoumarones, hydroxyisocoumarones, hydroxychalcones, hydroxychromones, anthocyans, quinones, hydroxyxanthones, 1,2-dihydroxybenzenes, 1,2,4-trihydroxybenzenes, 1,2,3-trihydroxybenzenes, 2,4,5-trihydroxytoluene, 5,6-dihydroxyindoline and mixtures thereof.

15. The composition according to claim 1, wherein the precursor of densification is chosen from among the extracts of plants, fruits, citrus fruits, vegetables and mixtures thereof.

16. The composition according to claim 1, wherein the precursor of densification is chosen from among the extracts of tea, grape, apple, banana, cacao, sorghum, potato and mixtures thereof.

17. The composition according to claim 1, wherein the precursor of densification is present in the ratio of at least 0.001% of the total weight of the composition.

18. The composition according to claim 1, wherein the physiologically acceptable medium is a medium making the precursor of densification soluble, in particular with a bacteriostatic property.

19. The composition according to claim 1, wherein the physiologically acceptable medium comprises at least one solvent of the precursor of densification.

20. The composition according to claim 1, wherein the physiologically acceptable medium comprises at least one solvent of the precursor of densification chosen from among water, the alcohols, polyols, polyol ethers and mixtures thereof.

21. The composition according to claim 1 wherein the physiologically acceptable medium comprises at least one alcohol chosen from among the alkanols and the alkanediols.

22. The composition according to claim 1, wherein the physiologically acceptable medium comprises an water/alcohol mixture.

23. The composition according to claim 22, wherein the alcohol represents up to 80% by weight of the mixture.

24. The composition according to claim 1, wherein the composition is free of any agent for chelation of the salt of Mn(II) and/or Zn(II).

25. The composition according to claim 1, wherein the composition is packaged in the form of an aerosol or a pump system without air uptake.

26. The composition according to claim 1, wherein the composition is in the form of two separate components, a first component comprising the catalytic system, dissolved in a physiologically acceptable medium, and a second component comprising the precursor of densification dissolved in a physiologically acceptable medium.

27. The composition according to claim 1, wherein the composition contains other ingredients chosen from among the thickeners or gelling agents with a hydro-alcoholic phase or an oily phase, coloring agents soluble in the medium of the composition, fillers, pigments, antioxidants, preservatives, fragrances, electrolytes, neutralizers, U.V. blocking agents, cosmetic and pharmaceutical active principles, mixtures thereof.

28. A process for densification of the human hair or for dressing of the hair, comprising applying to the hair and/or scalp a composition comprising, in a physiologically acceptable medium for topical application, an effective quantity of at least one precursor of densification, chosen from among the compounds comprising at least one aromatic cycle having at least two hydroxyl groups borne by two consecutive carbon atoms of the aromatic cycle, and an effective quantity of a catalytic system comprising a first constituent chosen from among the salts and oxides of Mn(II) and/or Zn(II) and mixtures thereof and a second constituent chosen from among the alkaline hydrogenocarbonates, the alkaline-earth hydrogenocarbonates and mixtures thereof, the proportions of the first and second constituents being such that:
[Mn(II)]/[HCO₃]≦1 with [Mn(II)]≠0
[Zn(II)]/[HCO₃]≦1 with [Zn(II)]≠0
[Mn(II)+Zn(II)]/[HCO₃]≦1 with [Mn(II) and [Zn(II)]≠0.
